(19) United States
(12) Patent Application Publication
(10) Pub. No.: US 2004/0099262 A1
(43) Pub. Date: May 27, 2004

(54) Emergency air supply worn as normal apparel

(76) Inventor: Russell Wolfe Otter, Lake Arrowhead, CA (US)

Correspondence Address: Russ Otter, 82612 Sky View Ln., Indio, CA, 92201, US

(21) Appl. No.: 10/373,323

(22) Filed: Feb. 24, 2003

Related U.S. Application Data
(60) Provisional application No. 60/428,974, filed on Nov. 25, 2002.

(51) Int. Cl. A61M 15/00

(52) U.S. Cl. 128/200.24

(57) ABSTRACT

Provides an emergency air supply system that is worn around the arm, presumably the wrist, and intended for use in air restrictive emergency conditions. Primarily it has been designed for the need to improve safety for water recreation or water sports where an individual is often caught gasping for air. This device is purposely designed to be no more obtrusive than a piece of clothing, such as a bathing suit, or a watch band would be, in order that it does not unduly restrict the water activity or water sport or any other potentially air restrictive activity you may engage in. The design is intended to encourage usage and increase safety, by way of its structural design that uses the strap-band itself as the mechanical structure of the entire mechanism. Thus creating a unique compact design, that is convenient to wear and provides air when you most need it.

### FIELD OF THE INVENTION

The present invention relates to an Emergency Air Device and, more particularly, to a portable and wearable emergency air system for providing an easily wearable device, that as well maybe detachable and held and operated in the mouth in and of itself. The new invention is designed to be no more obtrusive than a piece of clothing or a bathing suit might be, but with the added function of providing immediate oxygen/air availability. It could be worn on the arm, preferably the forearm and most probably the wrist. For the purpose of explanation in this patent I will concentrate my diagrams and explanation, using a Velcro wrist-band device, but any attachable strap device would work, be it snaps, hooks, and so on . . . The device could also be attached temporarily or permanently to any clothing or wearable equipment, such as the sleeve of a wet suit or even attached inside a watch-band.

Examples of usage: Any individual involved in a water recreation or water sport, such as body, wind or board surfing, sailing, rafting, kayaking, etc.; along with any such activity that may cause temporary air restriction or respiratory exhaustion. With these aforementioned conditions an individual who is body surfing in the ocean, for example, and who has lost control under the water in wave turbulence and is in need of air, could use the device, by either moving the arm to the mouth or quickly detaching the device and holding it the mouth, thus freeing up both arms to continue swimming to the surface for permanent air relief, thus avoiding a potential drowning and saving a life.

### BACKGROUND OF THE INVENTION

Emergency portable air devices have been in use for years. Typically, emergency portable air devices are comprised of a conventional, generally large manually hand-operated oxygen tank or tanks strapped to the body, (waist, or back), as in U.S. Pat. Nos.: 2002/0,153,009 A1, 5,529,061, 3,491,752, 2002/0,148,467 A1, 5,517,984,. Some emergency air systems maybe hand-held as well. All of these systems are either 1) Umbilically connected to the mouth via a hose and mouth piece device, or 2) are hand-held, or attached to other apparel. Consequently they are large and cumbersome, and not conducive to being worn without drawing clear and sometimes unwanted attention, unlike this proposed new invention, which is designed to be non-descript and worn as normal apparel, no less than a wrist watch would be.

These large body worn and umbilically mouth piece connected or hand-held devices do sometimes restrict water recreation and water sport activities. They would even be considered dangerous in certain adverse wave turbulence or rocky sea shore bottom conditions or in a river running a kayak near the rocks, or where you need all of your physical mobility, which the prior art devices could in some instances constrain and restrict physical mobility and even further endanger. Therefore they do not provide to a large community of water sport and recreation users the type of air safety devices they could use and are in need of today.

This new device provides for virtually complete physical mobility, unlike other devices, given the structural circumstances of the prior art presented in the previous paragraph. Further this new device is both usable in an attached arm mode held up to the mouth, or is also usable in a detached mode, held only by the mouth, whereby the air regulating system is designed to be controlled with the mouth and the teeth only, if desired.

This new emergency air device addresses the problem for everyone who has ever been caught gasping for air in an underwater or similar air restrictive situation. Most of us have experienced the discomfort and fear when caught under a river current or an oceans wave turbulence, or for that matter caught under the water in a swimming pool for any variety of reasons. This new device provides for the security of mind and critical safety, when engaged in a water activity or any activity that might create a condition whereby your air supply is restricted and you need immediate oxygen relief.

Primarily other prior art emergency air supply devices present themselves both structurally and operationally as to restricting, in size, or method of operation. In some cases other systems could also be unsafe, if surfing or kayaking near rocks that could snare and snag larger profile equipment causing a variety of safety concerns. This new device is designed to ameliorate or eliminate those consequences, and provide the user with a simple apparatus that does not unduly restrict activity, and therefore is easy and safe to use.

The only other device in prior art that I found through a formal patent search was a device that is similar in how it is applied to the mouth for medical purposes (Medication Dispenser) for nicotine or asthmatics, etc.—which is U.S. Pat. No. “6,233,744 B1”. This prior art device is fundamentally distinct from the device I am seeking a patent for—in Structure, Methodology of Use and Functional Purpose. It is similar in that is does not use an umbilical connection, nor is it hand held, and it uses the wrist to attach too.

It is dissimilar in the following:

1. Structurally U.S. Pat. No. “6,233,744 B1” only uses a STRAP to attach to the wrist and to the mechanical apparatus that supplies the aerosol Medication. In contrast—this new device structurally uses the STRAP as the mechanical structure in and of itself, as it houses the air and mechanical mouth regulator, as it wraps around the arm. To have only used the STRAP as an attachment mechanism in this new invention would have made the claims regarding “Non-Descript”, “Non-Obtrusive” and “Not Unduly Restrictive ” to the activity you were engaged in—invalid. This particular prior art device is contrary to those concepts.

2. The Prior Art embodied in U.S. Pat. No. “6,233,744 B1” also could not contain adequate liters of air necessary, as its compartments would be to small, without creating a large—very descript and bulbous mechanism on top of the wrist to manage the air volumes necessary, which is contrary to the safety and design intent embodied in this new invention. The structural design of all prior art in this field does not meet the low profile design and safety elements fashioned into this new design.

Consequently the Structural aspects are distinct opposites, i.e. one attaches the Arm and Aerosol Device to a Strap, and the Other Device is designed within the Strap, which provides additional operational distinctions as well.

3. The Prior Art in U.S. Pat. No. “6,233,744 B1” also does not function as an Air Supply Device; it only functions as a Medical Aerosol device. In all 17 of its claims it only refers to being used for “Aerosols”. This new patent is not an Aerosol Device, as “Aerosols”, use a propellant (usually an inert gas) and an active ingredient, liquid or particle (medication). This new invention is not an “Aerosol” at all by definition, as it only holds pressurized Air. Nor does this new device make any claims on being used as a medical supply aerosol dispenser. However structurally it would appear to be prima facie distinct, and I would surmise it therefore could be used in such a manner, given that a medical patient may want to have a more private less descript means of dispensing a medication in an emergency situation, in lieu of a more visual and obvious device, as represented in U.S. Pat. No. “6,223,744 B1”. That case, for medical purposes and converting this new device to an aerosol device being made, I am not seeking a patent for those purposes. Essential to the field of water sports and water recreation is the need for an emergency air device that can provide the attributes previously defined for a safe, ease of use device, thus encourage usage, to enhance both peace of mind and critical safety. The U.S. Pat. No. “6,233,744 B1” or any other prior art previously discovered does not meet, nor could they meet those criteria, structurally or functionally.

4. U.S. Pat. No. “6,223,744 B1” is also exclusively an electro-mechanical device, using microprocessors, circuit boards, and a battery power supply. This again, is in structural and operational contrast, to the new device in that the new device is not designed to operate as an electro-mechanical device. The new device is designed to operate mechanically without any power source, other than an individual's physical interactions, and thereby eliminates the critical points of failure endemic with electronic devices in a water based environment.

Summarizing the shortcomings of the Prior Art is as follows:

1. Large and cumbersome to wear and use. Thus does not encourage usage, reducing safety and additional loss of life.

2. In some situations, the previously large emergency equipment could snag on rocks or other impediments destroy the equipment and increase the safety risks.

3. Impedes and restricts activities, as the equipment is too large or cumbersome. (Usually either Hand-Held or Umbilically connected from tank to mouth.)

4. Other emergency air devices are not worn as normal everyday apparel, such as a watch-band, as this new apparatus would be, and therefore the prior art can draw unintended or undue attention.

5. Limited effectiveness in promoting and encouraging usage, through type of design and method of use.

6. In some cases, such as with U.S. Pat. No. “6,233,744 B1”, it is not used for, nor intended for use as an Air Supply device, and if it were, the design would not allow for enough air capacity, unless designed in a much larger more bulbous and more cumbersome manner, which would be problematic for safety and ease of use, and causing undue obstruction to movement in the water, along with safety concerns due to its bulbous wrist-top attachment design.

7. Not all emergency air systems or similar prior art are for use in a water based environment.

8. Prior Art to date does not encourage universal usage, which is a design objective of this new device.

It is therefore an object of the invention to promote safety, by encouraging emergency air device usage in all activity aspects that may cause air restriction.

It is another object of the invention to provide a tool that can reduce the level fear and anxiety both in the Emergency Air Device users mind and in the minds of parents and others who may be responsible for a child's or individuals welfare when simply swimming in a pool with friends or engaged in any activity that may create an restricted air environment.

It is another object of the invention to provide an Emergency Air System, which is desired by many, but yet unavailable in the market place.

It is another object of the invention to save lives, by providing air in a simple and seemingly natural, almost automatic manner, when you most need it.

### SUMMARY OF THE INVENTION

In view of the foregoing disadvantages inherent in the known types of “Emergency Air Supply Devices” now present in the prior art, the present invention provides a new wearable emergency air system construction that is neither an umbilically connected mouth piece nor hand-held emergency air system. In operation this device is simply worn around the arm presumably the wrist and is intended for use in air restrictive conditions. Once the device is held up to the mouth it is activated in the present design with the teeth squeezing the tubular, mushroom topped mouth piece which then regulates the air flow into the mouth.

Primarily it has been designed for the need to improve safety for water recreation or water sports were an individual is often caught gasping for air. This “Emergency Air Supply Device” provides the fix for that situation, were other such devices have not succeeded. Primarily because other devices are not conducive to use in highly physically demanding activities, such as wind, board, or body surfing, kayaking, sailing or rafting, or for that matter simply playing in the swimming pool, were for any variety of reasons your air becomes restricted. Fundamentally other “emergency air systems” structurally and operationally present themselves as to restricting, in size, or method of operation for use in very common or routine water recreation, water sport or other air restrictive routine activities. In some cases other systems could also be unsafe, if surfing or kayaking near rocks, that could snare and snag larger profile equipment causing a variety of safety concerns. This new device is designed to substantially reduce or eliminate those consequences, and provide the user with a simple apparatus, which does not unduly restrict your engaged in activity, and therefore is easy and safe to use. It provides air when you most need it. It is mechanically self-contained, worn on the arm presumably the wrist as any watch band would be worn. The design is intended to be wearable virtually at all times without looking out of place, i.e. worn as normal apparel. Given the devices intended ease of use—it should therefore encourage usage, and improve safety, saving lives and providing additional peace of mind to those responsible for individuals, or the individuals themselves, engaged in potentially air restrictive activities.

The working name for this new device is an acronym: “NOAH” which embodies several of its novel and unique characteristics:

Non Umbilically connected or Hand-Held

Oxygen—Air Supply used both in or out of water

Armband attached or detachable Mouth-Held

Housing specifically designed to not unduly restrict activity

### BRIEF DESCRIPTION OF THE DRAWINGS

A complete understanding of the present invention may be obtained by reference to the accompanying drawings, when considered in conjunction with the subsequent, detailed description, in which:

FIG. 1 This illustrates the Emergency Air Device as it would appear attached to the wrist, with the mouth piece depicted on the top section;

FIG. 2 Side view of the approximately half inch wide strap that would attach around the arm or wrist. Depicting the “single encasement air chamber”, mouth piece air regulation system, and air valve for air reloading from a conventional air pump; and

FIG. 3 Side view of the half inch wide strap that would attach around the arm or wrist. Depicting the “Multiple encasement air chamber” with air cylinders, mouth piece air regulation system, and air valve for air reloading from a conventional air pump.

For purposes of clarity and brevity, like elements and components will bear the same designations and numbering throughout the FIGURES.

### DESCRIPTION OF THE PREFERRED EMBODIMENT

Turning now descriptively to the drawings, in which similar reference characters denote similar elements throughout the different views. The attached figures illustrate an “Emergency Air Supply Device” that is worn on the arm, preferably the wrist, and is also detachable, as it could be held and operated independently from and by the mouth. The main components of this new invention include, an Arm/Wrist Strap Attached to the Wrist 10 that houses within the strap all of the major operational components. This structure is what makes this device unique and novel, in that all other prior art is housed as an attachment to a strap, or is hand-held, or is umbilically connected to an air source and no such arm or wrist band device is available for an emergency air supply. This embodiment depicted in the drawings utilizes the element of the Arm/Wrist Strap Attached to the Wrist 10 or potentially detached and held in the mouth independent of the arm—as the structural device in and of itself, thus utilizing critical space necessary for adequate air supplies, in conjunction with creating a device that is nearly non-obtrusive or encumbering to wear on your arm during a normal routine day, whether engaged in a potentially air restrictive activity or not.

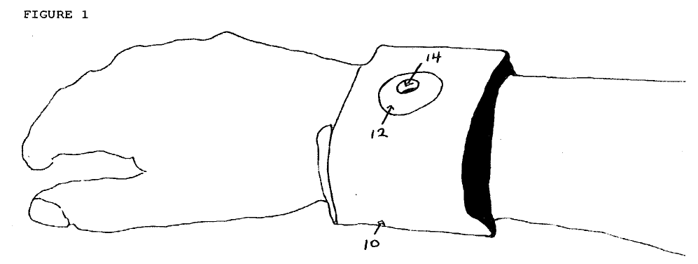

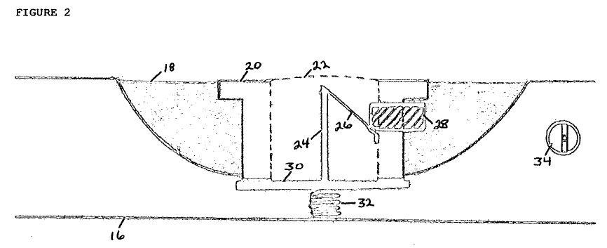

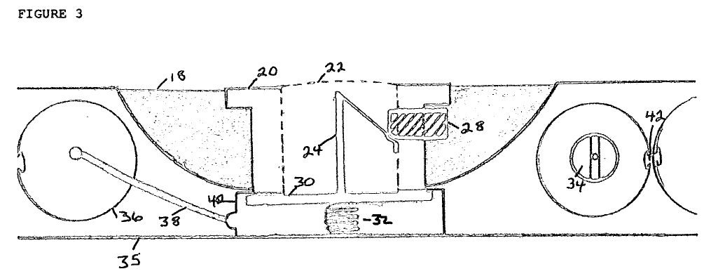

There are 3 drawings (FIGS.: 1, 2 & 3). FIG. 1 is a general overview of the device attached to a wrist. FIG. 2 and 3 depict two structurally different models. One model is seen in FIG. 2 and it is called the “Singular Air Encasement Strap 16 which is the simplest design, which uses the wrist band strap to encase the air. FIG. 3 depicts what is referred to as the “Multiple Air Cylinder Encasement Strap 35”, which uses several cylinder tubes linked together that contain the pressurized air supply.

Any type of attachable strap that can be effectively released in a quick manner would be appropriate, such as (Velcro, Snaps, Hooks, and so on.) The length of the arm or wrist strap is dependent on the size of the arm and the desired amount of air supply. For the purposes of the drawings and textual explanations I will use a Velcro Strap attached to the wrist. The Velcro will be part of an appropriate material that is designed to dry quickly, such as nylon or rayon, generally a synthetic material, which will house either of the two model designs: 1) Singular Air Encasement Strap 16 or 2) a Multiple Air Cylinder Encasement Strap 35. However the Singular Air Encasement Strap 16 uses a dense rubber or rubber composite, that is appropriate to contain at least 200 psi (pounds per square inch), and therefore the rubber containment shell, should be sufficient and not require any additional material. Contrary to that would be the Multiple Air Cylinder Encasement Strap 35, which uses metallic or non-metallic high pressure Air Cylinders 36, and should be capable of holding up to 1200 psi. The Air Cylinders 36 can be connected with a variety of means, dependent on the manufacturer. In the illustrated drawing figure (3)—I use the Cylinder Connection Links 42 made of metal with flanges on the ends that are sealed with rubber encased ends inside the Air Cylinders 36. The cylinders can vary in size, dependent on air requirements, but for an example, the Air Cylinders 36 would be approximately ½ inch in diameter and approximately 2 inches long, which should provide at 1200 psi approximately 10 liters of air or approximately 20 relaxed breaths. This would require a hand-pump that was hydraulic to refill. If you used the Singular Air Encasement Strap 16, with similar dimensions, you could easily hold up to 200 psi, at an estimated 2 liters or 4 relaxed breaths, using a standard hand-pump rated at 200 psi or greater to refill, using a conventional tire type Air Stem Valve 34, depicted in FIG. 2 and 3, but labeled in FIG. 2. The tire type Air Stem Valve 34 for air input in FIG. 2 is connected to the side of the rubber encasement. The tire type Air Stem Valve 34 in FIG. 3 is attached through the side of the material sleeve of the wrist strap and connected directly into one of the Air Cylinders 36.

Note: These size and capacity measurements listed in the paragraph above and below are intended as a frame of reference and not intended to be absolutes, as technologies and manufacturing size and capacity requirements may change.

The Mouth Piece 20 shown in all of the drawings could be a variety of types, using a variety of methods, to operate such as shifting the mouth, pulling pushing or turning to open and close the flow of air. The design chosen in these illustrated examples is a low profile nearly mushroomed shaped design, which can be operated solely by the mouth to regulate air without the assistance of leverage from the arm, so that the device could be held and operated in the mouth if detached from the arm. This is accomplished by securing the mouth around the t-shaped or mushroom shaped Mouth Piece 20 top which is approximately ¾ inch round on top surrounded by a 2″ diameter ⅜ Foam Shell around the Mouth Piece 18. The foam is not necessary, it simply provides a flush connection between the Strap, and Mouth Piece 20, that is easily depressible with the mouth and teeth. Once the mouth is secure around the mouth piece 20—then simply bite with the teeth on the Mouth On/Off Air Regulator Button 28 and the air is released into the mouth. The amount of air exhausted from the device is largely dependent on the degree by which the user depresses on the Mouth On/Off Air Regulator Button 28. When the Mouth On/Off Air Regulator Button 28 in the open position (depressed with the teeth), the Spring Rod 26 or other type of leveraging guide, pushes down on the Valve Stem 24, which opens the Air Seal 30 and then the Air flows up through the (FIG. 1) Air-Way 14 into the mouth. As the teeth release the Mouth On/Off Air Regulator Button 28, the Air Seal Spring 32 closes the Air Seal 30. In FIG. 2 which depicts the Singular Air Encasement Strap 16, the air is routed through the Air Seal 30 up through the Hollow Airway in the Mouth Piece 22 from the Hollow Singular Air Encasement Strap 16. In FIG. 3 which depicts the Multiple Air Cylinder Encasement Strap 35, the air flows from the Air Cylinders 36 through an appropriate high-pressure Air Hose 38, into the Air Release Chamber 40, and then up into the mouth, through the Hollow Airway in the Mouth Piece 22.

In operation this device is simply worn around the arm presumably the wrist and is intended for use in air restrictive conditions. Primarily it has been designed for the need to improve safety for water recreation or water sports were an individual is often caught gasping for air.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. An emergency air supply worn as normal apparel for providing emergency air in a compact structure, that is essentially unobtrusive and non-encumbering in design in order that it does not unduly limit physical activities being engaged in that may cause air restriction; as in its primary purpose to provide a device that increases safety when engaged in water recreation or water sport activities. Comprising:
Means for critical structural design attributes that allow for properly ample air capacities to be housed within the arm strap, whether the strap is independent or part of apparel, such as a wet suit sleeve. Additionally the method of operation, is based on a unique air supply device that works both in or out of water, that does not use umbilical connections nor is it hand held, in order that “immediate access” is not encumbered, by extra attachments or additional arm motions, beyond moving the arm to the mouth, and in some cases detaching the device to be operated independently by the mouth. This strap housing is fundamental in its design which does not attach air devices to the strap, but whereby the “Strap” is the air supply device in and of it self. This design further supports a low profile, small footprint device that enables a new “ease of use” in order to encourage usage, that should be safe to use in all routine and normal daily activities, and does not unduly restrict your specific water recreation or water-sport activity, such as a belt or back based umbilical air supply system would in many water recreation or water sport activities. For instance if you were body surfing, the additional drag of a waist-belt air tank and umbilical tube, and the added safety risk of hitting a rock or sea bottom with them, would generally tend to not encourage its use;
means for providing the air supply in a single dense rubber composite based or similarly appropriate lining;
means for opening and closing access to the emergency air supply, in a measured manner, dependent how much the air regulation button is depressed by the teeth or similar air regulation system. This design also is essential to allowing the mouth piece to be operated when detached from the arm, as it does not require any additional leverage from the arm to operate. In other words is designed to operate while being held by the mouth only;
means for providing additional air capacity attributed to high pressure cylinder encasements that connect to one another within the arm/wrist band strap; and
means for encasing the air cylinders and other operational components.

2. The emergency air supply worn as normal apparel in accordance with claim 1, wherein said means for critical structural design attributes, that allow for properly ample air capacities to be housed within the arm strap. Additionally the method of operation, is based on a unique air supply device that works both in or out of water, that does not use umbilical connections nor is it hand held, in order that “immediate access” is not encumbered, by extra attachments or additional arm motions, beyond moving the arm to the mouth, and in some cases detaching the device to be operated independently by the mouth. This strap housing is fundamental in its design which does not attach air devices to it, but is the air supply device in and of it self. this design further supports a low profile, small footprint device that enables a new “ease of use” in order to encourage usage, that should be safe to use in all routine and normal daily activities, and does not unduly restrict your specific recreational or water sport activity, such as a belt or back based umbilical air supply system would in many water recreation or water sport activities. For instance if you were body surfing, the additional drag of a waist-belt air tank and umbilical tube, and the added safety risk of hitting a rock or sea bottom with them, would generally tend to not encourage its use. Comprises an arm/wrist strap attached to the wrist or wrist apparel or is part of the wrist apparel.

3. The emergency air supply worn as normal apparel in accordance with claim 1, wherein said means for providing the air supply in a single dense rubber composite based lining. Comprises a singular air encasement strap.

4. The emergency air supply worn as normal apparel in accordance with claim 1, wherein said means for opening and closing access to the emergency air supply, in a measured manner, dependent how much the air regulation button is depressed by the teeth. This design also is essential to allowing the mouth piece to be operated when detached from the arm, as it does not require any additional leverage from the arm to operate. In other words is designed to operate while being held by the mouth only. Comprises a mouth on/off air regulator button.

5. The emergency air supply worn as normal apparel in accordance with claim 1, wherein said means for providing additional air capacity attributed to high pressure cylinder encasements that connect to one another within the arm/wrist band strap. Comprises air cylinders.

6. The emergency air supply worn as normal apparel in accordance with claim 1, wherein said means for encasing the air cylinders and other operational components comprises a multiple air cylinder encasement strap.

7. An emergency air supply worn as normal apparel for providing emergency air in a compact structure, that is essentially unobtrusive and non-encumbering in design in order that it does not unduly limit physical activities being engaged in that may cause air restriction; as in its primary purpose to provide a device that increases safety when engaged in water recreation or water sport activities. Comprising:
An arm/wrist strap attached to the wrist or obvious equivalent, for critical structural design attributes, which allow for properly ample air capacities to be housed within the arm strap. Additionally the method of operation, is based on a unique air supply device that works both in or out of water, that does not use umbilical connections nor is it hand held, in order that “immediate access” is not encumbered, by extra attachments or additional arm motions, beyond moving the arm to the mouth, and in some cases detaching the device to be operated independently by the mouth. This strap housing is fundamental in its design which does not attach air devices to it, but is the air supply device in and of it self. this design further supports a low profile, small footprint device that enables a new “ease of use” in order to encourage usage, that should be safe to use in all routine and normal daily activities, and does not unduly restrict your specific recreational or water sport activity, such as a belt or back based umbilical air supply system would in many water recreation or water sport activities. For instance if you were body surfing, the additional drag of a waist-belt air tank and umbilical tube, and the added safety risk of hitting a rock or sea bottom with them, would generally tend to not encourage its use;
a singular air encasement strap, for providing the air supply in a single dense rubber composite based lining;
a mouth on/off air regulator button, for opening and closing access to the emergency air supply, in a measured manner, dependent how much the air regulation button is depressed by the teeth. This design also is essential to allowing the mouth piece to be operated when detached from the arm, as it does not require any additional leverage from the arm to operate. In other words is designed to operate while being held by the mouth only;
an air cylinders, for providing additional air capacity attributed to high pressure cylinder encasements that connect to one another within the arm/wrist band strap; and
a multiple air cylinder encasement strap, for encasing the air cylinders and other operational components.

8. An emergency air supply worn as normal apparel for providing emergency air in a compact structure, that is essentially unobtrusive and non-encumbering in design in order that it does not unduly limit physical activities being engaged in that may cause air restriction; as in its primary purpose to provide a device that increases safety when engaged in water recreation or water sport activities. Comprising:
a Velcro, Snap, Hook, Tie Strap, Dense Rubber or Synthetic Nylon type material encasement dependent on single or multiple encasement design arm/wrist strap attached to the wrist, for critical structural design attributes, that allow for properly ample air capacities to be housed within the arm strap. additionally the method of operation, is based on a unique air supply device that works both in or out of water, that does not use umbilical connections nor is it hand held, in order that “immediate access” is not encumbered, by extra attachments or additional arm motions, beyond moving the arm to the mouth, and in some cases detaching the device to be operated independently by the mouth. This air strap housing is fundamental in its design which does not attach air devices to it, but is the air supply device in and of it self. This design further supports a low profile, small footprint device that enables a new “ease of use” in order to encourage usage, that should be safe to use in all routine and normal daily activities, and does not unduly restrict your specific water recreation or water sport activity, such as a belt or back based umbilical air supply system would in many water recreation or water sport activities. For instance if you were body surfing, the additional drag of a belt air tank and umbilical tube, and the added safety risk of hitting a rock or sea bottom with them, would generally tend to not encourage its use;
a Single Air Encasement singular air encasement strap, for providing the air supply in a single dense rubber composite based lining;
a foam shell around the mouth piece, for provides a flush design for the mouth piece in conjunction with the strap lining and mouth piece, along with establishing an easily depressible area for the mouth and teeth to encircle the mouth piece;
a Plastic, Metal, Wood, or any appropriate firm composite material mouth on/off air regulator button, for opening and closing access to the emergency air supply, in a measured manner, dependent how much the air regulation button is depressed by the teeth. This design also is essential to allowing the mouth piece to be operated when detached from the arm, as it does not require any additional leverage from the arm to operate. In other words is designed to operate while being held by the mouth only;
a metallic or plastic light weight composite material air cylinders, for providing additional air capacity attributed to high pressure cylinder encasements that connect to one another within the arm/wrist band strap; and
an appropriate material Liner multiple air cylinder encasement strap, for encasing the air cylinders and other operational components.
